(19) United States
(12) Patent Application Publication
(10) Pub. No.: US 2016/0304445 A1
(43) Pub. Date: Oct. 20, 2016

(54) N,N-BIS(2-AMINOALKYL)-1,2-ALKYL DIAMINE DERIVATIVES

(71) Applicant: BASF SE, Ludwigshafen (DE)

(72) Inventors: Stephanie JAEGLI, Mannheim (DE); Monika CHARRAK, Ludwigshafen (DE); Alexander PANCHENKO, Ludwigshafen (DE); Thomas SCHMIDT, Neustadt (DE); Johann-Peter MELDER, Boehl-iggelheim (DE)

(73) Assignee: BASF SE, Ludwigshafen (DE)

(21) Appl. No.: 15/101,964

(22) PCT Filed: Nov. 21, 2014
(86) PCT No.: PCT/EP14/75263

§ 371 Date: Jun. 6, 2016

(30) Foreign Application Priority Data
Dec. 11, 2013 (EP) 13196613.7

(51) Int. Cl. C07C 265/04; C07C 209/48; C07C 263/10; C07C 211/14

(52) U.S. Cl. CPC C07C 265/04; C07C 211/14; C07C 209/48; C07C 263/10

(57) ABSTRACT

The present invention relates to 2-N,N-(bis-2-aminoalkyl)-1,2-alkyldiamine derivatives of formula (I) as such, to a process for preparing the 2-N,N-(bis-2-aminoalkyl)-1,2-alkyldiamine derivatives, to the use of the 2-N,N-(bis-2-aminoalkyl)-1,2-alkyldiamine derivatives as hardeners for epoxy resins, as an intermediate in the preparation of triisocyanates, as initiators for polyetherols and/or as monomers for the preparation of polyamides, to triisocyanates derived from the 2-N,N-(bis-2-aminoalkyl)-1,2-alkyldiamine derivatives of formula (I) and also to a process for preparing these triisocyanates.

The present invention relates to 2-N,N-(bis-2-aminoalkyl)-1,2-alkyldiamine derivatives of formula (I) as such

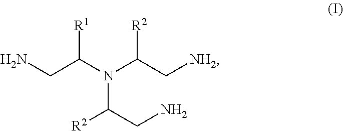

to a process for preparing the 2-N,N-(bis-2-aminoalkyl)-1,2-alkyldiamine derivatives, to the use of the 2-N,N-(bis-2-aminoalkyl)-1,2-alkyldiamine derivatives as hardeners for epoxy resins, as an intermediate in the preparation of triisocyanates, as initiators for polyetherols and/or as monomers for the preparation of polyamides, to triisocyanates derived from the 2-N,N-(bis-2-aminoalkyl)-1,2-alkyldiamine derivatives of formula (I) and also to a process for preparing these triisocyanates.

It is widely known that aliphatic nitriles can be hydrogenated in the presence of hydrogen and catalysts to form the corresponding amines. Such hydrogenation processes are known for both β-aminonitriles and various α-aminonitriles, such as aminoacetonitrile (AAN) or ethylenediaminediacetonitrile (EDDN), for the preparation corresponding amines, such as ethylenediamine (EDA) or triethylenetetramine (TETA). It is also known that the hydrogenation of β-anninonitriles generally proceeds without problems, whereas the hydrogenation of α-anninonitriles is associated with the occurrence of numerous disadvantages, such as the hydrogenolysis of the C—CN bond of the nitrile used and/or the H₂N—C bond of the amine obtained by the hydrogenation. The “Handbook of Heterogeneous Catalytic Hydrogenation for Organic Synthesis” (John Wiley & Sons, New York, 2001, pages 173-275) illustrates the problematic nature of the hydrogenation of α-aminonitriles with reference to cyclic α-aminonitriles such as 1-cyclohexyl-2,5-dicyano-2,5-dimethylpyrrolidine.

EP 382508 describes a process for hydrogenating nitrilotriacetonitrile (NTAN) as a batch process. Continuous processes are explicitly excluded in this document.

WO 2008/080755 likewise describes the hydrogenation of NTAN. However, this hydrogenation is carried out in the presence of ammonia since according to WO 2008/080755 primary amines are only obtainable in this manner.

U.S. Pat. No. 8,227,641 describes the preparation of polyfunctional amines by hydrogenation of the corresponding polyfunctional nitriles, wherein the hydrogenation is carried out in the presence of alcohol, water or a water/alcohol mixture and ammonia.

WO 2008/104553 relates to a process for preparing triethylenetetraamine (TETA), wherein ethylenediaminediacetonitrile (EDDN) is hydrogenated in the presence of a catalyst and a solvent. Furthermore, EDDN can also be present as a component of an aminonitrile mixture which additionally contains ethylenediaminemonoacetonitrile (EDMN), wherein diethylenetriamine (DETA) is obtained from EDMN by hydrogenation. TETA and DETA are both non-cyclic (linear) ethyleneamines.

The corresponding methylglycinenitrile-N,N-diacetonitrile (referred to herein as MGDN) used in the process according to the invention for preparing 2-N,N-(bis-2-aminoethyl)-1,2-propane-diamine (referred to herein as MGTA) is already known from U.S. Pat. No. 5,849,950. Said document describes the preparation of MGDN as such, wherein alpha-alaninenitrile is reacted with formaldehyde and hydrocyanic acid (HCN). A possible hydrogenation of the product obtained in this process to obtain the corresponding aminomethyl compound is, however, not disclosed in this document. Numerous further documents are known from the prior art, which all describe processes for preparing MGDN, such as, for example, EP1881957. However, the hydrogenation of MGDN to form MGTA is not described or suggested in any of the documents.

EP 375279 describes the preparation of alkyleneamines formed by ring-opening of aziridines. According to formula (III) of EP 375279, the inventive 2-N,N-(bis-2-aminoalkyl)-1,2-alkyldiamine derivatives of formula (I) would also fall under formula (III) of EP 375279 in the case where in formula (III) R¹=methyl or ethyl, R²═H, R³ and R⁴═C₂₋₄ aminoalkyl groups and n=1. However, it is known from Yusin, A. et al. in “Aziridines and Epoxides in Organic Synthesis, ED-Wiley-VCH; Weinheim, Germany 2006” and also from Chernitskij K. et al in Zhurnal Obshchei Khimii, 60(3), 617-25; 1990 under “Nucleophilic Cleavage and Formation of Saturated Heterocycles. X. Reactivity of 2-methylaziridine in Aminolysis Reactions” that in the ring-opening reactions nucleophilic attack occurs at the sterically least-hindered C atom so that in formula (III) of EP 375279, R¹ is preferably hydrogen and correspondingly R² is methyl or ethyl. Therefore, the inventive 2-N,N-(bis-2-aminoalkyl)-1,2-diaminoalkyl derivatives of formula (I) are not obtainable from the process described in EP 375279.

U.S. Pat. No. 3,527,757 likewise discloses the reaction of aziridine rings with primary or secondary amines. However, the inventive 2-N,N-(bis-2-aminoalkyl)-1,2-alkyldiamines of formula (I) are not obtainable by this process since it is to be expected that the attack occurs at the sterically least-hindered carbon and thus the target product according to the invention is not obtained.

Compounds having two or three primary amino functions (“diamines” or “triamines”) can be used for numerous applications, for example as hardeners in epoxy resins or for preparing diisocyanates or triisocyanates. The structure of the polyamine used can influence the properties of the polymer materials prepared from the polyamines, such as resistance to weathering, resistance to hydrolysis, resistance to chemicals, light stability, electrical properties and also mechanical properties. However, it can also exert an influence on the processability and the processing of the polyamines to form the corresponding polymer materials, for example the curing of epoxy resins.

It is therefore an object of the present invention to provide compounds having three primary amino functions, which can be used for hardening epoxy resins and which exhibit new property profiles in the preparation of polyamines. A further object of the invention is to provide a process that enables the selective preparation in high yield of 2-N,N-(bis-2-aminoalkyl)-1,2-alkyldiamines alkylated in the 2-position such as 2-N,N-(bis-2-aminoethyl)-1,2-propanediamine (MGTA), because using the known propyleneimine ring opening the preferably obtainable products are N,N-(bis-2-aminoalkyl)-1,2-alkyldiamines maximally alkylated in the 1-position which are the regioisomers of the compounds according to the invention.

Furthermore, the production of troublesome by-products such as 2-piperazin-1-yl-propan-1-amine is to be reduced, particularly in the preparation of MGTA.

This object is achieved by 2-N,N-(bis-2-aminoalkyl)-1,2-alkyldiamine derivatives of general formula (I)

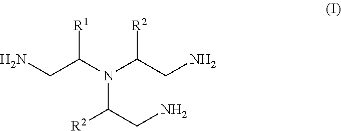

where
R¹ is selected from the group consisting of branched or unbranched aliphatic C₁₋₁₀ alkyl groups and
R² is selected from the group consisting of hydrogen and branched or unbranched aliphatic C₁₋₁₀ alkyl groups.

It is advantageous when in the inventive compounds of formula (I), R¹ is selected from the group consisting of methyl, ethyl, propyl, isopropyl, n-butyl, sec-butyl and tert-butyl and R² is selected from the group consisting of hydrogen, methyl, ethyl, propyl, isopropyl, n-butyl, sec-butyl, and tert-butyl.

It is advantageous when in the inventive compounds of formula (I), R¹ is methyl and R² is hydrogen.

The invention further provides a process for preparing the compounds of formula (I), wherein the 2-N,N-(bis-2-aminoalkyl)-1,2-alkyldiamine derivatives are prepared from the corresponding 2-[N,N-(bis-1-cyanoalkyl)amino]alkylnitriles of formula (II)

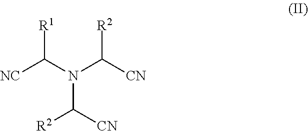

where
R¹ is selected from the group consisting of branched or unbranched aliphatic C₁₋₁₀ alkyl groups and
R² is selected from the group consisting of hydrogen and branched or unbranched aliphatic C₁₋₁₀ alkyl groups
   by hydrogenation in the presence of a catalyst and hydrogen.

It is advantageous in the process according to the invention when R¹≠H and the hydrogenation is carried out as a continuous operation in the absence of ammonia.

The invention further provides a process for preparing the compounds of formula (I), wherein the 2-N,N-(bis-2-aminoalkyl)-1,2-alkyldiamine derivatives are prepared from the corresponding 2-[N,N-(bis-1-cyanoalkyl)amino]alkylnitriles of formula (IIa)

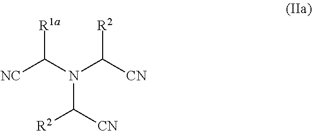

where
R^{1a} is selected from the group consisting of branched or unbranched aliphatic C₁₋₁₀ alkyl groups and hydrogen and
R² is selected from the group consisting of hydrogen and branched or unbranched aliphatic C₁₋₁₀ alkyl groups
   by hydrogenation in the presence of a catalyst and hydrogen, wherein the process is carried out as a continuous operation in the absence of ammonia.

The two processes according to the invention are advantageous when a catalyst which comprises as active species one or more elements selected from the group consisting of Fe, Co, Ni, Ru, Rh, Pd, Os, Ir and Pt is used.

The two processes according to the invention are advantageous when a Raney nickel catalyst or a Raney cobalt catalyst is used as catalyst.

The two processes according to the invention are advantageous when the catalyst used is a Raney cobalt catalyst which comprises at least one of the elements Mo, Cr or Fe as promoter.

The two processes according to the invention are advantageous when the hydrogenation is carried out in a solvent selected from the group consisting of amides, aromatic hydrocarbons, alcohols, amines, esters and ethers.

The two processes according to the invention are advantageous when the hydrogenation is carried out at temperatures in the range from 60 to 180° C. and at pressures in the range from 40 to 300 bar.

The two processes according to the invention are advantageous when methylglycinenitrilediacetonitrile (MGDN) is hydrogenated to form N,N-(bis-2-aminoethyl)-1,2-propanediamine (MGTA).

The two processes according to the invention are advantageous when methylglycinenitrilediacetonitrile (MGDN) and NTAN are used in pure crystalline form.

The invention further provides the use of one of the 2-N,N-(bis-2-aminoalkyl)-1,2-alkyldiamine derivatives of formula (I) as a hardener for epoxy resins, as an intermediate in the preparation of triisocyanates, as an initiator in the preparation of polyetherols and/or as a monomer for the preparation of polyamides.

The invention further provides triisocyanates according to the general formula (III)

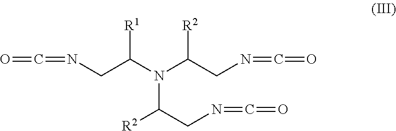

where
R¹ is selected from the group consisting of branched or unbranched aliphatic C₁₋₁₀ alkyl groups and hydrogen and
R² is selected from the group consisting of hydrogen and branched or unbranched aliphatic C₁₋₁₀ alkyl groups.

It is advantageous when in the triisocyanates according to the invention, R¹ is methyl and R² is hydrogen.

The present invention further provides a process for preparing the triisocyanate according to the invention, wherein a 2-N,N-(bis-2-aminoalkyl)-1,2-alkyldiamine derivative of formula (I) is reacted with phosgene.

In the 2-N,N-(bis-2-aminoalkyl)-1,2-alkyldiamines of formula (I)

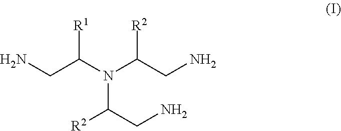

where
R¹ is selected from the group consisting of branched or unbranched aliphatic C₁₋₁₀ alkyl groups and
R² is selected from the group consisting of hydrogen and branched or unbranched aliphatic C₁₋₁₀ alkyl groups,

R¹ is preferably selected from the group consisting of methyl, ethyl, n-propyl, i-propyl, n-butyl, sec-butyl and tert-butyl, methyl and ethyl are particularly preferred, methyl particularly so.

In formula (I), R² is preferably selected from the group consisting of hydrogen, methyl, ethyl, n-propyl, i-propyl, n-butyl, sec-butyl and tert-butyl; hydrogen, methyl and ethyl are particularly preferred, hydrogen particularly so.

Preferred compounds are selected from the group in which R¹ is methyl and R² is hydrogen, R¹ is methyl and R² is methyl, and R¹ is methyl and R² is ethyl; the compound in which R¹ is methyl and R² is hydrogen (N,N-(bis-2-aminoethyl)-1,2-propanediamine (described herein as MGTA)) is particularly preferred.

The process according to the invention for preparing the compounds of formula (I) is generally carried out by reacting the compounds of formula (II),

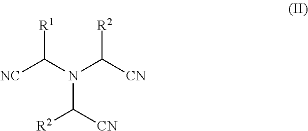

wherein R¹ and R² are as defined above, with hydrogen in the presence of a hydrogenation catalyst. It is preferred that the hydrogenation process in which compounds of formula (II) are used is carried out as a continuous operation without ammonia.

In a particular embodiment of the above process, the hydrogenation is carried out in the absence of ammonia and as a continuous operation and in this case the compounds of formula (IIa) are used.

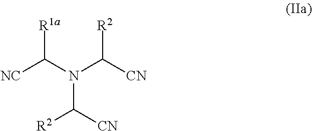

where
R^{1a} is selected from the group consisting of branched or unbranched aliphatic C₁₋₁₀ alkyl groups and hydrogen and
R² is selected from the group consisting of hydrogen and branched or unbranched aliphatic C₁₋₁₀ alkyl groups.

However, it is advantageous when the compounds of formula (II) are used in the process.

The inventive 2-N,N-(bis-2-aminoalkyl)-1,2-alkyldiamine derivatives of formula (I) can be prepared in an advantageous manner with high conversion and/or high selectivity. By-products, such as, for example, the corresponding 2-piperazin-1-ylalkyl-1-amine derivative or the 2-(2,6-dialkyl)piperazin-1-ylalkyl-1-amine derivative are formed only in small amounts and can be further minimized by controlling reaction parameters such as pressure, temperature or catalyst. In particular, the hydrogenation of MGDN to obtain the corresponding N,N-(bis-2-aminoethyl)-1,2-propanediamine as such (MGTA) can be carried out with high selectivity of preferably 60%, more preferably >65%.

In the hydrogenation to form the 2-N,N-(bis-2-aminoalkyl)-1,2-alkyldiamine derivative according to the invention, at least six mol of hydrogen per mol of N,N-(bis-2-cyanoalkyl)1,2-alkyldinitriles are generally required. However, the process can also be carried out using an excess of hydrogen.

The temperatures at which the hydrogenation is carried out are generally in the range from 60 to 180° C., preferably from 80 to 140° C., in particular at 100 to 130° C.

The prevailing pressure in the hydrogenation is generally 40 to 300 bar, preferably 40 to 240 bar, more preferably 80 to 200 bar.

In a preferred embodiment, the compounds of formula (II) or (IIa) are fed into the hydrogenation at a rate not greater than the rate at which the compound of formula (II) or (IIa) reacts with hydrogen in the hydrogenation.

The feed rate should preferably be adjusted so that full conversion is achieved. This is influenced by temperature, pressure, type of compound of formula (II) or (IIa), amount and type of catalyst and of reaction medium, mixing quality of the reactor contents, residence time etc.

The process according to the invention is carried out in the presence of a catalyst. The catalysts used can in principle be all of the catalysts known to those skilled in the art for a nitrile hydrogenation. The catalysts used for hydrogenating the three nitrile functions to form the inventive compounds of formula (II) or (IIa) can therefore be, for example, catalysts comprising as active species one or more elements of transition group 8 (Fe, Co, Ni, Ru, Rh, Pd, Os, Ir, Pt), preferably Fe, Co, Ni, Ru or Rh, more preferably Co or Ni.

Included therein are oxidic catalysts, which comprise one or more active species in the form of their oxygen compounds, and skeletal catalysts (also known as Raney® type; below also: Raney catalyst) obtained by leaching (activation) of an alloy composed of hydrogenation-active metal and a further component (preferably Al). The catalysts may additionally comprise one or more promoters.

In a particularly preferred embodiment, Raney catalysts, preferably Raney cobalt or Raney nickel catalysts and more preferably a Raney cobalt catalyst comprising at least one of the elements Mo, Cr or Fe as promoter are used in the hydrogenation of compounds of formula (II) or (IIa). The Raney cobalt catalyst is thus doped with at least one of the elements Mo, Cr or Fe.

The catalysts can be used as unsupported catalysts or in supported form. Metal oxides such as Al₂O₃, SiO₂, ZrO₂, TiO₂, mixtures of metal oxides or carbon (activated carbons, carbon blacks, graphite) are preferably used as supports.

Before use, the oxidic catalysts are activated outside the reactor or in the reactor by reduction of the metal oxides at elevated temperature in a hydrogen-containing gas stream. When the catalysts are reduced outside the reactor, subsequent passivation by an oxygen-containing gas stream or by embedding in an inert material can be carried out in order to avoid uncontrolled oxidation in air and to enable safe handling. Organic solvents such as alcohols but also water or an amine, preferably the reaction product, can be used as the inert material. Skeletal catalysts, which can be activated by leaching with aqueous base as described, for example, in EP-A 1 209 146 are an exception in the activation step. Depending on the process carried out (suspension hydrogenation, fluidized-bed process, fixed-bed hydrogenation), the catalysts are used in the form of powder, spall or shaped bodies (preferably extrudates or tablets).

Particularly preferred fixed bed catalysts are the unsupported cobalt catalysts doped with Mn, P and alkali metal (Li, Na, K, Rb, Cs) and disclosed in EP-A1 742 045. The catalytically active material of these catalysts consists, before the reduction with hydrogen, of 55 to 98 wt %, in particular 75 to 95 wt %, of cobalt, 0.2 to 15 wt % of phosphorus, 0.2 to 15 wt % of manganese and 0.05 to 5 wt % of alkali metal, in particular sodium, in each case calculated as the oxide.

Further useful catalysts are the catalysts disclosed in EP-A 963 975, the catalytically active material of which comprises before the treatment with hydrogen 22 to 40 wt % of ZrO₂, 1 to 30 wt % of oxygen compounds of copper, calculated as CuO, 15 to 50 wt % of oxygen compounds of nickel, calculated as NiO, where the molar Ni: Cu ratio is greater than 1, 15 to 50 wt % of oxygen compounds of cobalt, calculated as CoO, 0 to 10 wt % of oxygen compounds of aluminum and/or manganese, calculated as Al₂O₃ and MnO₂ respectively, and no oxygen compounds of molybdenum, for example the catalyst A disclosed in this document with the composition of 33 wt % of Zr, calculated as ZrO₂, 28 wt % of Ni, calculated as NiO, 11 wt % of Cu, calculated as CuO, and 28 wt % of Co, calculated as CoO.

Further useful catalysts are those disclosed in EP-A 696 572, the catalytically active material of which comprises before the reduction with hydrogen 20 to 85 wt % of ZrO₂, 1 to 30 wt % of oxygen compounds of copper, calculated as CuO, 30 to 70 wt % of oxygen compounds of nickel, calculated as NiO, 0.1 to 5 wt % of oxygen compounds of molybdenum, calculated as MoO₃, and 0 to 10 wt % of oxygen compounds of aluminum and/or manganese, calculated as Al₂O₃ and MnO₂ respectively. For example, the catalyst disclosed specifically in this document with the composition of 31.5 wt % of ZrO₂, 50 wt % of NiO, 17 wt % of CuO and 1.5 wt % of MoO₃. Equally useful are the catalysts described in WO-A-99/44984 comprising (a) iron or a compound based on iron or mixtures thereof, (b) 0.001 to 0.3 wt %, based on (a), of a promoter based on 2, 3, 4 or 5 elements selected from the group consisting of Al, Si, Zr, Ti, V, (c) 0 to 0.3 wt %, based on (a), of a compound based on an alkali metal and/or alkaline earth metal and also d) 0.001 to 1 wt %, based on (a), of manganese.

For suspension processes, Raney catalysts are preferably used. With the Raney catalysts, the active catalyst is prepared as a “metal sponge” composed of a binary alloy (nickel, iron, cobalt, with aluminum or silicon) by leaching out one partner with acid or alkali. Residues of the original alloy partner often act synergistically.

The Raney catalysts used in the process according to the invention are preferably prepared starting from an alloy of cobalt or nickel, with particular preference given to cobalt, and a further alloy component which is soluble in alkalis. Aluminum is preferably used as this soluble alloy component, but other components such as zinc and silicon or mixtures of such components can also be used.

To activate the Raney catalyst, the soluble alloy component is completely or partially extracted with alkali for which, for example, sodium hydroxide can be used. The catalyst can subsequently be washed, for example with water or organic solvents.

One or more further elements can be present as promoters in the catalyst. Examples of promoters are metals of transition groups IB, VIB and/or VIII of the Periodic Table, such as chromium, iron, molybdenum, nickel, copper, etc. The activation of catalysts by leaching of the soluble component (typically aluminum) can be carried out either in the reactor itself or prior to introduction into the reactor. The preactivated catalysts are air-sensitive and pyrophoric and are therefore generally stored and handled under a medium such as, for example, water, an organic solvent or a material which is present in the reaction according to the invention (solvent, starting material, product) or embedded in an organic compound which is solid at room temperature.

In a preferred embodiment and in accordance with the invention, a Raney cobalt skeletal catalyst which was obtained from a Co/Al alloy by leaching with aqueous alkali metal hydroxide solution, for example sodium hydroxide, and subsequent washing with water and which comprises at least one of the elements Fe, Ni or Cr as promoters is used.

Such catalysts typically also comprise in addition to cobalt, 1-30 wt % of Al, particularly 2-12 wt % of Al, very particularly 3-6 wt % of Al, 0-10 wt % of Cr, particularly 0.1-7 wt % of Cr, very particularly 0.5-5 wt % of Cr, in particular 1.5-3.5 wt % of Cr, 0-10 wt % of Fe, particularly 0.1-3 wt % of Fe, very particularly 0.2-1 wt % of Fe, and/or 0-10 wt % of Ni, particularly 0.1-7 wt % of Ni, very particularly 0.5-5 wt % of Ni, in particular 1-4 wt % of Ni, where the weight data are in each case based on the total weight of the catalyst.

A “Raney 2724” cobalt skeletal catalyst from W.R. Grace & Co., for example, may advantageously be used in the process according to the invention. This catalyst has the following composition:

### Al: 2-6 wt %, Co: ≧86 wt %, Fe: 0-1 wt %, Ni: 1-4 wt %, Cr: 1.5-3.5 wt %.

Likewise, according to the invention it is possible to use a nickel skeletal catalyst obtained from an Ni/Al alloy by leaching with aqueous alkali metal hydroxide solution, for example sodium hydroxide solution, and subsequent washing with water, which catalyst comprises as a promoter at least one of the elements Fe and Cr.

Such catalysts typically also comprise in addition to nickel 1-30 wt % of Al, particularly 2-20 wt % of Al, very particularly 5-14 wt % of Al, 0-10 of wt % Cr, particularly 0.1-7 wt % of Cr, very particularly 1-4 wt % of Cr, and/or 0-10 wt % of Fe, particularly 0.1-7 wt % of Fe, very particularly 1-4 wt % of Fe, where the weight data are in each case based on the total weight of the catalyst.

In the event of declining activity and/or selectivity, the catalysts can optionally be regenerated using the methods known to those skilled in the art as disclosed, for example, in WO99/33561 and the documents cited therein.

The regeneration of the catalysts can be carried out in the actual reactor (in situ) or on the catalyst after removal from the reactor (ex situ). In fixed-bed processes, regeneration is preferably carried out in situ and in suspension processes part of the catalyst is preferably removed as a continuous operation or batchwise, regenerated ex situ and returned.

The process according to the invention can be carried out in the presence of a solvent. All solvents known to those skilled in the art may in principle be used as the solvent, with solvents that are inert towards the compounds of formula (II) or (IIa) being preferred.

Possible solvents are organic solvents, for example amides, such as N-methylpyrrolidone (NMP) and dimethylformamide (DMF), aromatic and aliphatic hydrocarbons, such as toluene, alcohols such as methanol, ethanol, propanol, isopropanol, butanol, isobutanol, secondary butanol and tertiary butanol, amines, such as EDA or ethylamines and ammonia, esters, such as methyl acetate or ethyl acetate and ethers, such as diisopropyl ether, diisobutyl ether, glycol dimethyl ether, diglycol dimethyl ether, dioxane and tetrahydrofuran (THF).

The solvent is preferably an amide, an aromatic hydrocarbon, an alcohol, an amine, an ester or an ether. Ethers are more preferably used in the process according to the invention, even more preferably cyclic ethers and particular preference is given to tetrahydrofuran.

The solvent is normally used in a weight ratio of 0.1:1 to 15:1 to the compounds of formula (II) or (IIa) used. The concentration of the compounds of formula (II) or (IIa) in the solution in which the hydrogenation is carried out should be selected such that a suitable feed rate and/or residence time can be set. It is preferable to mix the compounds of formula (II) or (IIa) in an amount of 5 to 50 wt % with the solvent. Based on the particularly preferred solvent tetrahydrofuran, it is, for example, advantageous to use the compounds of formula (II) or (IIa) in amount of 10 to 40 wt % based on the solvent.

The reaction of the compounds of formula (II) or (IIa) with hydrogen in the presence of catalysts can be carried out as a continuous operation, as a semicontinuous operation or batchwise in a fixed-bed, fluidized-bed or suspension mode in conventional reaction vessels suitable for catalysis. Reaction vessels in which contacting of the compounds of formula (II) or (IIa) and the catalyst with the hydrogen under pressure is possible, are useful for carrying out the hydrogenation.

The hydrogenation in suspension mode can be carried out in a stirred reactor, jet loop reactor, jet nozzle reactor, bubble column reactor or in a cascade of identical or different reactors of this type.

The hydrogenation over a fixed-bed catalyst preferably takes place in one or more tube reactors or else shell-and-tube reactors.

The hydrogenation of the nitrile groups takes place with release of heat, which generally has to be removed. The heat can be removed by installed heat transfer surfaces, cooling jackets or external heat exchangers in a circuit around the reactor. The hydrogenation reactor or a cascade of hydrogenation reactors can be operated in a single pass. Alternatively, a circulation mode is also possible, in which part of the reactor output is recycled to the reactor inlet, preferably without preceding workup of the circulation stream.

In particular, the circulation stream can be cooled in a simple and inexpensive manner by means of an external heat exchanger, and the heat of reaction can thus be removed.

The reactor can also be operated adiabatically. In the case of adiabatic operation of the reactor, the temperature rise in the reaction mixture can be limited by cooling the feeds or by supplying “cold” organic solvent.

Since in that case the reactor itself need not be cooled, a simple and inexpensive design is possible. One alternative is that of a cooled shell-and-tube reactor (only in the case of a fixed bed). A combination of the two modes is also conceivable. In this case, a fixed bed reactor is preferably connected downstream of a suspension reactor.

The catalyst may be arranged in a fixed bed (fixed bed mode) or suspended in the reaction mixture (suspension mode).

In a particularly preferred embodiment, the catalyst is suspended in the reaction mixture to be hydrogenated.

The settling rate of the hydrogenation catalyst in the solvent selected should be low, meaning that it does not settle within 10 seconds, in order that the catalyst can be readily kept in suspension. The particle size of the catalysts used in suspension mode is therefore preferably from 0.1 to 500 μm, in particular from 1 to 100 μm.

If the hydrogenation of the compounds of formula (II) or (IIa) is performed as a continuous operation in suspension mode, the compounds of formula (II) or (IIa) are preferably continuously supplied to the reactor and a stream comprising the hydrogenation products (N,N-(bis-2-aminoalkyl)-1,2-alkyldiamine derivatives) is continuously removed from the reactor.

In the case of the batchwise suspension mode, a semibatchwise mode with supply of the compounds of formula (II) or (IIa) is preferred.

The amount of catalyst is preferably 1 to 60 wt %, more preferably 5 to 40 wt % and yet more preferably 20 to 30 wt %, based on the overall reaction mixture.

The residence time in the reactor in the case of the batchwise suspension mode is preferably 0.1 to 6 hours, more preferably 0.5 to 2 hours.

The residence time in the reactor in the case of the continuous suspension mode is preferably 0.1 to 6 hours, more preferably 0.5 to 2 hours.

The space velocity over the catalyst in the case of the continuous suspension mode and in the semibatchwise process is 0.1 to 5 kg, preferably 0.2 to 2 kg and more preferably 0.3 to 1 kg of compounds of formula (II) or (IIa) per kg of catalyst and hour.

If the reaction is carried out in suspension mode in a stirred reactor, the power input via the stirrer is preferably 0.1 to 100 kW per m³.

Spent catalyst can be removed by filtration, centrifugation or crossflow filtration. It may also be necessary to compensate for losses of original catalyst amount resulting from attrition and/or deactivation by adding fresh catalyst.

Following the hydrogenation, the output from the hydrogenation can optionally be purified further. The catalyst can be removed by methods known to those skilled in the art. Generally, the hydrogen present during the hydrogenation is removed after removal of the catalyst.

Hydrogen is preferably removed by lowering the pressure at which the hydrogenation was carried out to a value at which hydrogen is gaseous, but the other components in the reaction output are present in the liquid phase. The reaction output is preferably decompressed into a vessel from a hydrogenation pressure of preferably 60 to 325 bar, more preferably 100 to 280 bar and most preferably 170 to 240 bar, to a pressure of 5 to 50 bar. At the top of the vessel, hydrogen, any ammonia, and also as the case may be a small amount of evaporated low boilers or solvents, such as THF, are obtained.

Any organic solvents present in the reaction output are generally likewise removed by distillation. In particular, the inventive N,N-(bis-2-aminoalkyl)-1,2-alkyldiamine derivatives can be isolated from the reaction product by methods known to those skilled in the art.

The present invention also relates to the use of the compounds of formula (I) as a hardener for epoxy resins, as an intermediate in the preparation of triisocyanates, as an initiator in the preparation of polyetherols and/or as a monomer for the preparation of polyamides.

The compounds of formula (I) are alternative hardeners for epoxy resins, they present new opportunities in the formulation and processing of epoxy resins and they can be used for regulating the property spectrum of epoxy resins. The inventive compounds of formula (I) show fast curing times and high glass transition temperatures as hardeners for epoxy resins which makes them particularly useful for use in adhesives, floor coatings and resin transfer molding (RTM) applications.

The compounds of formula (I) can also be used as an intermediate in the preparation of the corresponding triisocyantates of the general formula (III) set out below.

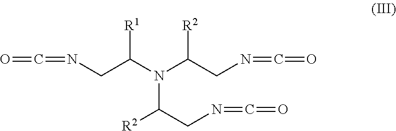

In general formula (III), the respective radicals R¹ and R² are as defined above for the general formulae (I).

These triisocyanates are useful for the preparation of light-stable polyurethanes, for example as a paint/varnish or coating, and on account of their structure offer new formulation possibilities and thus access to novel and interesting property profiles. These triisocyanates are obtainable, for example, by reacting compounds of formula (I) with phosgene.

The compounds of formula (I) can also be used as initiators in the preparation of polyetherols. The compounds of formula (I) are CH acidic compounds which can be deprotonated with a base and onto which alkylene oxides, such as ethylene oxide, propylene oxide and/or butylene oxide can subsequently be added.

Alkyloxylated triamines can, for example, be used as catalysts in PU preparation.

The compounds of formula (I) can be used as monomers in the preparation of polyamides. Thus, the compounds of formula (I) can be reacted, for example, with dicarboxylic acids, such as, for example, succinic acid, adipic acid, terephthalic acid and/or phthalic acid to form polymers.

Furthermore, the present invention therefore also provides a triisocyanate according to general formula (III),

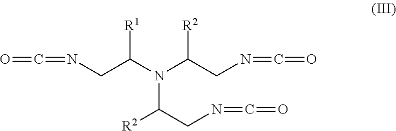

wherein R¹ and R² are as defined in formula (I) and the preferred and particularly preferred variants are also the same as for R¹ and R² from formula (I).

The present invention therefore also further provides a process for preparing a triisocyanate according to one of the above described definitions.

Processes for preparing triisocyanates from the corresponding triamines, for example by reaction with phosgene, are known to those skilled in the art and these processes may therefore be correspondingly applied by those skilled in the art.

According to the invention the triisocyanates are preferably prepared by reacting a corresponding compound of formula (I) with phosgene. It is preferable to use an N,N-(bis-2-aminoethyl)-1,2-propanediamine (MGTA) in this reaction and mixtures of two or more such compounds of formula (I) may also optionally be used.

The invention is illustrated below with the aid of examples.

### EXAMPLE 1

### Preparation of N,N-(bis-2-aminoethyl)-1,2-propanediamine (Semibatchwise)

Into a 270 ml autoclave equipped with baffles and a disk stirrer, 5 g of (dry) Raney cobalt and 40 g of THF were initially charged. The autoclave was heated to 120° C. and hydrogen was injected to an overall pressure of 100 bar. A mixture of 10 g of pure MGDN in 90 g of THF was metered in over a period of 2 h. The reaction mixture was stirred for a further 60 minutes under the reaction conditions. The conversion and the selectivity were determined by GC analysis (GC column: DB1, length=30 m, internal diameter=0.32 mm, film thickness=1 μm−80° C. starting temperature and temperature ramp 10° C./min to 280° C.−carrier gas=helium−FID detector) and quoted in % by area.

The hydrogenation output comprised 61% of N,N-(bis-2-aminoethyl)-1,2-propanediamine, 15% of the below listed piperazine derivatives of formula (1), 7.5% of the below listed aminopiperazine derivatives of formula (2) and 8% of the below listed dehydropiperazine derivatives of formula (3). The remainder was unknown secondary components.

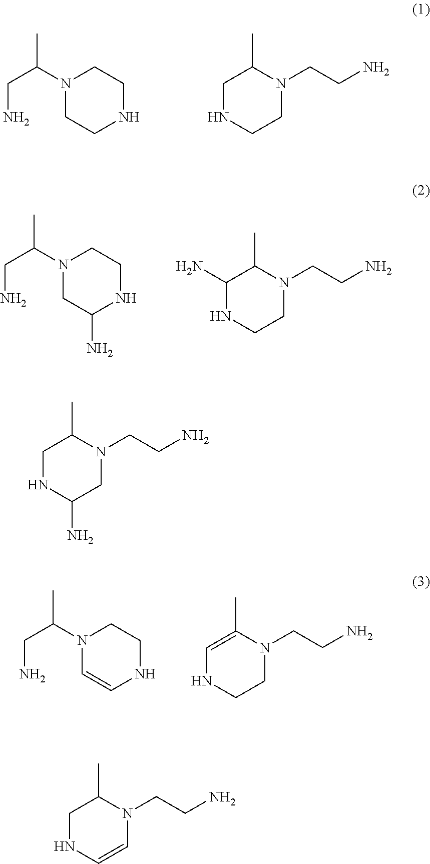

### EXAMPLE 2

### Preparation of N,N-(bis-2-aminoethyl)-1,2-propanediamine (Continuous)

Into a 270 ml autoclave equipped with baffles and a disk stirrer, 7 g (dry) of Raney cobalt and 0.1 g of 25% aqueous sodium hydroxide solution in 40 g of THF were initially charged. 15 l (STP)/h of hydrogen were continuously supplied. 50 g per hour of a mixture of 10 g of pure MGDN in 90 g of THF were continuously pumped in at 190 bar. The temperature in the reactor was 120° C. The catalyst was separated from the reactor output by continuous filtration through a sintered metal frit having a pore diameter of 500 nm. The output was decompressed through a regulating valve. Hydrogen was subsequently removed in a downstream phase separator. Under these conditions, 100% conversion was achieved and the yield of N, N-(bis-2-aminoethyl)-1,2-propanediamine was 70% including the product content in the crude output. Altogether 300 g of MGDN were used and the selectivity remained constant over this 60 h period of time. In addition to the product according to the invention (70% MGTA), the hydrogenation output comprised 6% of the compounds of formula (1) above, 5.5% of the compounds of formula (2) above and 4% of the compounds of formula (3) above. The remainder of the hydrogenation output was unknown secondary components.

Part of the crude reaction mixture was concentrated in a rotary evaporator and distilled through a Vigreux column at <0.5 mbar. The product was distilled off overhead at 95° C. The product N,N-(bis-2-aminoethyl)-1,2-propanediamine was obtained in a purity of 85%.

The product was characterized by GC-MS and NMR.

¹³C NMR (125 MHz, THF): 58.73, 53.73, 46.37, 41.85, 31.25.

GC-MS: DB1 column, 30 m, 0.32 mm, 1 μm; 80° C. starting temperature, temperature ramp 10° C./min to 280° C.-retention time 10.11 min (93.3% by area). The GC-MS conditions are identical to those in Example 1.

Example relating to use in epoxy resin systems

### EXAMPLE 3

### Preparation of the Reactive Resin Composition and Investigation of the Reactivity Profile

The formulations to be compared with one another were prepared by mixing stoichiometric amounts of the amine with a bisphenol-A-diglycidyl ether-based epoxy resin (EEW 182), and investigated immediately.

The rheological measurements for investigating the reactivity profile of the amines with epoxy resins were carried out at different temperatures on a shear rate-controlled plate-plate rheometer (MCR 301, Anton Paar) having a plate diameter of 15 mm and a gap distance of 0.25 mm.

Investigation 3a) Comparison of the time required for the freshly prepared reactive resin composition to attain a viscosity of 10 000 mPa*s at a defined temperature: the measurement was carried out in rotation on the aforementioned rheometer at different temperatures (23° C. and 75° C.).

Investigation 3b) Comparison of the gelling times: the measurement was carried out in rotation-oscillation on the aforementioned rheometer at 23° C. and 75° C. The point of intersection of loss modulus (G″) and storage modulus (G′) gives the gelling time.

| | a | | | | | |
|---|---|---|---|---|---|---|
| | Viscosity increase to 10 000 mPas | | | | b | |
| | 23° C. | | | | Isothermal | |
| | Starting | | 75° C. | | gelling time | |
| | viscosity | | Starting | | 23° C. | 75° C. |
| 3 | Ø 2-5 min | Time | viscosity | Time | Time | Time |
| Name | [mPas] | [min] | [mPas] | [min] | [min] | [min] |
| D230 | 886 | 478 | 39 | 40 | 1850 | 60 |
| T403 | 304 | 1269 | 60 | 39 | 2090 | 52 |
| TETA | 330 | 109 | 182 | 6.5 | 325 | 16 |
| MGTA | 620 | 72 | 206 | 6 | 195 | 10.5 |

### Result

MGTA shows an extremely short gelling time compared with other aliphatic amines polyetheramine D230, polyetheramine T403 and triethylenetetraamine (TETA).

### EXAMPLE 4

### Exothermic Profile of the Reactive Resin Composition and Glass Transition Temperatures of the Cured Thermosets

The DSC investigation of the curing reaction of the amines with a bisphenol-A-diglycidyl ether-based epoxy resin (EEW 182) for determining onset temperature (Tₒ), exothermicity (ΔE), and also determining the glass transition temperatures (T_{g}) were carried out according to ASTM D 3418.

Investigation 4) Analysis program for the DSC investigations: 0° C.→5 K/min 180° C.→30 min 180° C.→20 K/min 0° C.→20 K/min 220° C.

| 4 | | | |
|---|---|---|---|
| Name | Onset [° C.] | ΔH [J/g] | Tg [° C.] |
| D230 | 83.3 | 421.9 | 93 |
| T403 | 84.3 | 399.3 | 90.3 |
| TETA | 63.1 | 586.8 | 151.6 |
| MGTA | 61.2 | 587.1 | 153.4 |

### Result

MGTA shows an extremely high glass transition temperature compared to other aliphatic amines.

1. A 2-N,N-(bis-2-aminoalkyl)-1,2-alkyldiamine derivative of general formula (I): 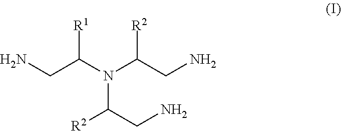
where
R¹ is selected from the group consisting of at least one branched or unbranched aliphatic C₁₋₁₀ alkyl groups and
R² is selected from the group consisting of at least one of hydrogen and branched or unbranched aliphatic C₁₋₁₀ alkyl groups.

2. The compound of formula (I) according to claim 1, wherein R¹ is selected from the group consisting of at least one of methyl, ethyl, propyl, isopropyl, n-butyl, sec-butyl and tert-butyl and R² is selected from the group consisting of at least one of hydrogen, methyl, ethyl, propyl, isopropyl, n-butyl, sec-butyl and tert-butyl.

3. The compound of formula (I) according to claim 1, wherein R¹ is methyl and R² is hydrogen.

4. A process for preparing a compound of formula (I), wherein the 2-N,N-(bis-2-aminoalkyl)-1,2-alkyldiamine derivative is prepared from the corresponding 2-[N,N-(bis-1-cyanoalkyl)amino]alkylnitrile of formula (II) by hydrogenation in the presence of a catalyst and hydrogen,
wherein formula (II) is: 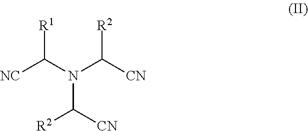
where
R¹ is selected from the group consisting of at least one branched or unbranched aliphatic C₁₋₁₀ alkyl groups and
R² is selected from the group consisting of at least one of hydrogen and branched or unbranched aliphatic C₁₋₁₀ alkyl groups.

5. The process according to claim 4, wherein the hydrogenation is carried out as a continuous operation in the absence of ammonia.

6. A process for preparing a compound of formula (I), wherein the 2-N,N-(bis-2-aminoalkyl)-1,2-alkyldiamine derivative is prepared from the 2-[N,N-(bis-1-cyanoalkyl)amino]alkylnitrile of formula (IIa) by hydrogenation in the presence of a catalyst and hydrogen, wherein the process is carried out as a continuous operation in the absence of ammonia: wherein formula (IIa) is: 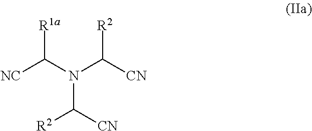
where
R^{1a} is selected from the group consisting of at least one branched or unbranched aliphatic C₁₋₁₀ alkyl groups and hydrogen and
R² is selected from the group consisting of at least one of hydrogen and branched or unbranched aliphatic C₁₋₁₀ alkyl groups.

7. The process according to claim 4, wherein said catalyst comprises as active species one or more elements selected from the group consisting of Fe, Co, Ni, Ru, Rh, Pd, Os, Ir and Pt.

8. The process according to claim 4, wherein the catalyst is a Raney nickel catalyst or a Raney cobalt catalyst.

9. The process according to claim 4, wherein the catalyst is a Raney cobalt catalyst which comprises at least one of the elements Mo, Cr and Fe as promoter.

10. The process according to claim 4, wherein the hydrogenation is carried out in a solvent selected from the group consisting of amides, aromatic hydrocarbons, alcohols, amines, esters and ethers.

11. The process according to claim 4, wherein the hydrogenation is carried out at temperatures in the range from 60 to 180° C. and at pressures in the range from 40 to 300 bar.

12. The process according to claim 4, wherein methylglycinenitrile-N,N-diacetonitrile (MGDN) is hydrogenated to form N,N-bis(2-aminoethyl)-1,2-propanediamine (MGTA).

13. The process according to claim 12, wherein methylglycinenitrile-N,N-diacetonitrile (MGDN) is used in pure crystalline form.

14. A method for making a hardener for epoxy resins, an intermediate in the preparation of diisocyanates, an initiator in the preparation of polyetherols, as or a monomer for the preparation of polyamides comprising incorporating or providing a N,N-(bis-2-aminoalkyl)-1,2-alkyldiamine derivatives of formula (I) according to claim 1.

15. A triisocyanate according to general formula (III): 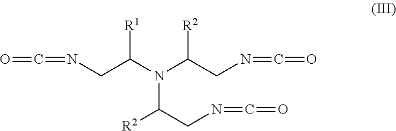
where
R¹ is selected from the group consisting of at least one branched or unbranched aliphatic C₁₋₁₀ alkyl groups and hydrogen and
R² is selected from the group consisting of at least one of hydrogen and branched or unbranched aliphatic C₁₋₁₀ alkyl groups.

16. The triisocyanate according to claim 15, wherein R¹ is methyl and R² is hydrogen.

17. A process for preparing a triisocyanate according to general formula (III), wherein a 2-N,N-(bis-2-aminoalkyl)-1,2-alkyldiamine derivative of formula (I) according to claim 1 is reacted with phosgene.

18. The process according to claim 6, wherein said catalyst comprises as active species one or more elements selected from the group consisting of Fe, Co, Ni, Ru, Rh, Pd, Os, Ir and Pt.

19. The process according to claim 6, wherein the catalyst is a Raney nickel catalyst or a Raney cobalt catalyst.

20. A hardener for an epoxy resin, an intermediate for preparation of a diisocyanate, an initiator for preparation of a polyetherol, or a monomer for preparation of a polyamide comprising a N,N-(bis-2-aminoalkyl)-1,2-alkyldiaminederivative of formula (I) according to claim 1.
